(19) United States
(12) Patent Application Publication
(10) Pub. No.: US 2018/0194859 A1
(43) Pub. Date: Jul. 12, 2018

(54) Linked Dual Antibodies Conjugated at a Hinge Region

(71) Applicant: Sorrento Therapeutics, Inc., San Diego, CA (US)

(72) Inventors: Gunnar F. Kaufmann, San Diego, CA (US); Yanwen Fu, San Diego, CA (US); James Patterson, Haverstraw, NY (US); Tong Zhu, San Diego, CA (US)

(73) Assignee: Sorrento Therapeutics, Inc., San Diego, CA (US)

(21) Appl. No.: 15/870,830

(22) Filed: Jan. 12, 2018

Related U.S. Application Data
(60) Provisional application No. 62/445,290, filed on Jan. 12, 2017.

(51) Int. Cl. C07K 16/46

(52) U.S. Cl. CPC C07K 16/46; A61K 2039/505

(57) ABSTRACT

There is disclosed methods for derivatizing and conjugating two hinge-containing antibody molecules. There is further disclosed derivatized and conjugated hinge-containing antibody molecules.

### CROSS REFERENCE TO RELATED APPLICATION

The present patent application claims priority to U.S. provisional patent application 62/445,290 filed 12 Jan. 2017.

### TECHNICAL FIELD

There is disclosed derivatized and conjugated Linked Dual Antibodies (LDALD) and methods for making the same.

### BACKGROUND

Bispecific antibodies (LDALD) provide binding to two antigens simultaneously but are a single antibody structure. Initial efforts at developing LDAb antibodies were focused on recruiting T-cells to a target, and monovalent antigen binding and elimination of FcγR binding. Eliminating FcγR binding avoids antigen-independent T-cell activation, which, in turn, can lead to side effects such as cytokine release syndrome. Although such LDAb formats (e.g., BiTE or DART) are effective at recruiting T-cells, they can suffer from low avidity and decreased serum half-life.

Antibody engineering efforts have produced LDAb formats based on the native immunoglobulin structure in order to avoid avidity loss and/or reduced serum half-life. Knob-into-hole, DUOBODY, and CrossMAb technologies provide the advantage of containing an antibody Fc region but still only enable T-cell recruitment by monovalent antigen binding. Alternative formats, such as dual variable domain immunoglobulin (DVD-Ig) and scFv/Fv-mAb fusion LDALD, target multiple tumor-associated antigens or multiple epitopes on a single antigen in a multi-valent manner and often rely on effector functions or delivery of a drug payload for cytotoxicity. However, expression and purification of these formats can frequently present challenges that become a barrier to development.

LDALD have also been produced by conjugation of reduced F(ab′) monomers with 5,5′-dithiobis (2-nitrobenzoic acid) and o-phenylenedimaleimide at hinge cysteines to make F(ab′)₂ fragments. More recently, a dual homobifunctional dibromomaleimide (DBM) linker was used to synthesize αHER×αCEA Fab-scFv LDAb by disulfide bridging. However, a drawback to using a bis-DBM linker is the need to use excess linker to avoid homodimerization. Accordingly, there is a need in the art for a novel LDAb platform that allows for the generation of of single or dual antibodies from native, pre-existing monoclonal antibodies and that have binding specificities to two different targets. The present disclosure was made to address this need in the art.

### SUMMARY

The present disclosure provides a method for functionalizing a hinge-containing antibody molecule, comprising:

(a) reducing an antibody interchain disulfide bond in a hinge region of an antibody molecule having at least two interchain disulfide bonds, to produce two sulfhydryl groups in reduced interchain disulfide bonds; and

(b) contacting the antibody molecule with two sulhydryls with a linker, wherein the linker comprises a sulfhydryl-reactive moiety and a bioorthogonal moiety, whereby each of the sulfhydryl groups of the reduced interchain disulfide bond are conjugated to the sulfhydryl-reactive moiety to form a covalently-linked adduct with the hinge; wherein after contacting the hinge-containing molecule with the linker, the hinge has at least one interchain disulfide bond. Preferably, the antibody molecule is selected from the group consisting of IgG2 and a F(ab′)₂. Preferably, reducing is accomplished by using a reducing agent selected from the group consisting of: tris(2-carboxyethyl)phosphine (TCEP), dithiothreitol (DTT), mercaptoethanol, mercaptoethylamine, cysteine, glutathione, thiophenol, and benzeneselenol. Most preferably, the reducing agent is dithiothreitol (DTT). Preferably, the sulfhydryl-reactive moiety comprises a dibromomaleimide (DBM) moiety. Preferably, the bioorthogonal moiety comprises a click chemistry handle, comprising one or more moieties selected from the group consisting of: an azide; a nitrone; a cyclooctyne; an aldehyde; a ketone; a tetrazine; a cyclooctene; an isonitrile; a quadracyclane; a nickel bis(dithiolene), and a dibenzylcyclooctyne (DBCO).

The present disclosure provides a method for conjugating two hinge-containing antibody molecules, comprising:

(a) reducing an interchain disulfide bond in a first hinge of a first hinge-containing antibody molecule to produce two sulfhydryls;

(b) contacting the first hinge with a first linker comprising a first sulfhydryl-reactive moiety and a first bioorthogonal moiety, such that each of the sulfhydryl groups of the reduced first hinge is conjugated to the first sulfhydryl-reactive moiety to form a covalently-linked adduct with the first hinge-containing molecule;

(c) reducing an interchain disulfide bond in a second hinge of a second hinge-containing antibody molecule to produce two sulfhydryls; contacting the second hinge with a second linker comprising a second sulfhydryl-reactive moiety and a second bioorthogonal moiety, such that each of the sulfhydryl groups of the reduced second hinge is conjugated to the second sulfhydryl-reactive moiety to form a covalently-linked adduct with the second hinge-containing molecule; and

(d) contacting the first and second hinge-containing antibody molecules such that the first and second bioorthogonal moieties form a covalently-linked adduct between the first hinge-containing molecule and the second hinge-containing molecule, wherein after contacting the first and second hinge-containing molecule with the first and second linker, the first and second hinge each has at least one interchain disulfide bond. Preferably, the first or second hinge-containing antibody molecule is selected from the group consisting of a full-length antibody, an IgG2, and a F(ab′)₂. Preferably, the reducing agent is selected from the group consisting of: tris(2-carboxyethyl)phosphine (TCEP), dithiothreitol (DTT), mercaptoethanol, mercaptoethylamine, cysteine, glutathione, thiophenol, and benzeneselenol. Preferably, the sulfhydryl-reducing moiety comprises a dibromomaleimide (DBM) moiety. Preferably, the bioorthogonal moiety comprises a click chemistry handle, wherein the click chemistry handle comprises one or more moieties selected from the group consisting of: an azide; a nitrone; a cyclooctyne; an aldehyde; a ketone; a tetrazine; a cyclooctene; an isonitrile; a quadracyclane; a nickel bis(dithiolene), and a dibenzylcyclooctyne (DBCO).

The present disclosure provides an IgG2 class antibody or F(ab′)2 fragment thereof, having a hinge region hinge-containing molecule comprising: a hinge comprising an interchain disulfide bond; and a linker molecule comprising a hinge-joining moiety that covalently links both cysteine residues of a disulfide bond-forming cysteine pair of the hinge, and a bioorthogonal moiety, wherein, the hinge has at least one interchain disulfide bond.

The present disclosure provides a composition comprising: a first hinge-containing antibody molecule comprising a first hinge and an interchain disulfide bond; a second hinge-containing antibody molecule comprising a second hinge and an interchain disulfide bond; and a linker molecule comprising a first hinge-joining moiety that covalently links both cysteine residues of a disulfide bond-forming cysteine pair of the first hinge, a second hinge-joining moiety that covalently links both cysteine residues of a disulfide bond-forming cysteine pair of the second hinge, and a bioorthogonal moiety that covalently links the first and second hinge-containing molecules.

### BRIEF DESCRIPTION OF THE FIGURES

FIG. 1 is a schematic diagram of a method for conjugating two native antibodies into a single LDAb antibody.

FIG. 2A is a chemical structure for a linker used in conjugating native antibodies (dibromomaleimide-PEG4-azide).

FIG. 2B is a chemical structure for a linker used in conjugating native antibodies (dibromomaleimide-PEG4-DBCO).

FIG. 3A is a chromatogram for SEC HPLC of LDAb created using TCEP as a reducing agent. Peaks for the LDAb, anti-EGFR antibody, and anti-HER2 antibody are indicated by the arrows.

FIG. 3B is a chromatogram for SEC HPLC of LDAb created using DTT as a reducing agent.

FIG. 3C is a chromatograph of purified LDAb, using SEC HPLC.

FIG. 4 is a series of HIC HPLC chromatograms of untreated trastuzumab IgG2, untreated cetuximab IgG2, trastuzumab IgG2 azide, cetuximab IgG2-DBCO, the click reaction, and the purified LDAb.

FIG. 5 depicts SDS-PAGE non-reducing and reducing gels, with lanes labeled for untreated trastuzumab IgG2, untreated cetuximab IgG2, trastuzumab IgG2 azide, cetuximab IgG2-DBCO, the click reaction, and the purified LDAb.

FIG. 6A is a surface plasmon resonance (SPR) graph for binding of the HER2 antigen by trastuzumab IgG2 (upper graph) and the LDAb (lower graph).

FIG. 6B is a surface plasma resonance graph for binding of the EGFR antigen to cetuximab IgG2 (upper graph) and the LDAb (lower graph).

FIG. 7 is a biolayer interferometry graph of a bispecific antibody binding HER2 antigen and EGFR antigen over time.

FIG. 8A is a bar graph showing levels of HER2 expression of various cancer cell lines (HCC1954, MDA-MB-468, MCF7, and MDA-MB-435), as detected by immunofluorescent staining and high throughput flow cytometry.

FIG. 8B is a bar graph showing levels of EGFR expression of various cancer cell lines (HCC1954, MDA-MB-468, MCF7, and MDA-MB-435), as detected by immunofluorescent staining and high throughput flow cytometry.

FIG. 8C is an HTFC graph of mean fluorescence intensity for staining of the cancer cell line HCC1954 with trastuzumab IgG2, cetuximab IgG2, and the LDAb conjugated from trastuzumab IgG2 and cetuximab IgG2.

FIG. 8D is an HTFC graph of mean fluorescence intensity for staining of the cancer cell line MDA-MB-468 with trastuzumab IgG2, cetuximab IgG2, and the LDAb conjugated from trastuzumab IgG2 and cetuximab IgG2.

FIG. 8E is an HTFC graph of mean fluorescence intensity for staining of the cancer cell line MCF7 with trastuzumab IgG2, cetuximab IgG2, and the LDAb conjugated from trastuzumab IgG2 and cetuximab IgG2.

FIG. 8F is an HTFC graph of mean fluorescence intensity for staining of the cancer cell line MDA-MB-435 with trastuzumab IgG2, cetuximab IgG2, and the LDAb conjugated from trastuzumab IgG2 and cetuximab IgG2.

### DETAILED DESCRIPTION

Provided herein are novel methods for derivatizing and conjugating two hinge-containing molecules (e.g., antibodies). Also provided are derivatized and conjugated hinge-containing molecules (e.g., LDAb antibodies). The methods and compositions disclosed herein are particular useful for the rapid production of LDAb antibodies from two native, pre-existing monoclonal antibodies.

### I. Definitions

The terms “antibody” and “antibodies” include full length antibodies, antigen-binding fragments of full length antibodies, and molecules comprising antibody CDRs, VH regions or VL regions. Examples of antibodies include monoclonal antibodies, recombinantly produced antibodies, monospecific antibodies, multispecific antibodies (including bispecific antibodies), multivalent (including bivalent, trivalent, tetravalent, etc.) antibodies, human antibodies, humanized antibodies, chimeric antibodies, murine antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies (e.g. scFv-Fc) or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. In a specific embodiment, the antibody is a humanized monoclonal antibody. In another specific embodiment, the antibody is a human monoclonal antibody.

Antibody light and heavy chains are divided into regions of structural and functional homology. The term “region” refers to a part or portion of an immunoglobulin or antibody chain and includes constant region or variable regions, as well as more discrete parts or portions of said regions. For example, light chain variable regions include “complementarity determining regions” or “CDRs” interspersed among “framework regions” or “FRs”, as defined herein.

The regions of an immunoglobulin heavy or light chain may be defined as “constant” (C) region or “variable” (V) regions, based on the relative lack of sequence variation within the regions of various class members in the case of a “constant region”, or the significant variation within the regions of various class members in the case of a “variable regions”. The terms “constant region” and “variable region” may also be used functionally. In this regard, it will be appreciated that the variable regions of an immunoglobulin or antibody determine antigen recognition and specificity. Conversely, the constant regions of an immunoglobulin or antibody confer important effector functions such as secretion, transplacental mobility, Fc receptor binding, complement binding, and the like. The subunit structures and three-dimensional configurations of the constant regions of the various immunoglobulin classes are well known.

The constant and variable regions of immunoglobulin heavy and light chains are folded into domains. The term “domain” refers to a globular region of a heavy or light chain comprising peptide loops (e.g., comprising 3 to 4 peptide loops) stabilized, for example, by β-pleated sheet and/or intrachain disulfide bond. Constant region domains on the light chain of an immunoglobulin are referred to interchangeably as “light chain constant region domains”, “CL regions” or “CL domains”. Constant domains on the heavy chain (e.g. hinge, CH1, CH2, or CH3 domains) are referred to interchangeably as “heavy chain constant region domains”, “CH” region domains or “CH domains”. Variable domains on the light chain are referred to interchangeably as “light chain variable region domains”, “VL region domains or “VL domains”. Variable domains on the heavy chain are referred to interchangeably as “heavy chain variable region domains”, “VH region domains” or “VH domains”.

By convention, the numbering of the variable constant region domains increases as they become more distal from the antigen binding site or amino-terminus of the immunoglobulin or antibody. The N-terminus of each heavy and light immunoglobulin chain is a variable region and at the C-terminus is a constant region; the CH3 and CL domains actually comprise the carboxy-terminus of the heavy and light chain, respectively. Accordingly, the domains of a light chain immunoglobulin are arranged in a VL-CL orientation, while the domains of the heavy chain are arranged in the VH-CH1-hinge-CH2-CH3 orientation.

Amino acid positions in a heavy chain constant region, including amino acid positions in the CH1, hinge, CH2, CH3, and CL domains, may be numbered according to the Kabat index numbering system (see Kabat et al., in “Sequences of Proteins of Immunological Interest”, U.S. Dept. Health and Human Services, 5th edition, 1991). Alternatively, antibody amino acid positions may be numbered according to the EU index numbering system (see Kabat et al, id.)

The term “VH domain” refers to the amino terminal variable domain of an immunoglobulin heavy chain, and the term “VL domain” refers to the amino terminal variable domain of an immunoglobulin light chain.

The term “CH1 domain” includes the first (most amino terminal) constant region domain of an immunoglobulin heavy chain that extends, e.g., from about positions 114-223 in the Kabat numbering system (EU positions 118-215). The CH1 domain is adjacent to the VH domain and amino terminal to the hinge region of an immunoglobulin heavy chain molecule, and does not form a part of the Fc region of an immunoglobulin heavy chain.

The term “hinge region” refers to the portion of a heavy chain molecule that joins the CH1 domain to the CH2 domain, or a variant, or a cysteine residue-containing fragment thereof. This hinge region comprises approximately 25 residues and is flexible, thus allowing the two N-terminal antigen binding regions to move independently. Hinge regions can be subdivided into three distinct domains: upper, middle, and lower hinge domains (Roux et al. J. Immunol. 1998, 161:4083). The hinge region also includes cysteine residues that allow formation of covalent disulfide bonds. In an intact native antibody, the free sulfhydryl groups of cysteine residues on each of the two heavy chains form a covalent disulfide bond, joining the two heavy chains.

The term “hinge” refers to a dimer of two hinge regions that are linked together by at least one disulfide bond between a disulfide bond-forming cysteine pair.

The term “hinge-containing molecule” refers to a molecule that includes a hinge.

The term “disulfide bond-forming cysteine pair” within the context of a hinge refers to a pair of cysteine residues (one from each of the two hinge regions constituting a hinge) that react together under physiological conditions to form an interchain disulfide bond.

The term “interchain disulfide bond” within the context of a hinge refers to a disulfide bond formed between the sulfydryl groups of the cysteine residues in a disulfide bond-forming cysteine pair in a hinge.

The term “non-hinge disulfide bonds” refers to disulfide bonds in a hinge-containing molecule that are not within the hinge. For example, non-hinge disulfide bonds include, but are not limited to: disulfide bonds between the light chains and heavy chains of the variable region; intrachain disulfide bonds between cysteine residues of the heavy chain in the variable region of an antibody or Fab fragment; intrachain disulfide bonds between cysteine residues of the light chain in the variable region of an antibody or Fab fragment; and intrachain disulfide bonds between cysteine residues in the heavy chains of the constant region.

The number of intermolecular disulfide bonds between monomeric subunits of wild-type Fc molecules ranges from 1 to 11 depending on class (e.g., IgG, IgA, and IgE) or subclass (e.g., IgG1, IgG2, IgG3, IgA1, and IgGA2). For example, IgG1 may have two cysteine residues in the hinge region of each heavy chain, forming two disulfide bonds between the two heavy chains, while IgG2 may have four cysteine residues in each hinge region to form four disulfide bonds between the two heavy chains. IgG3 may have eleven disulfide bonds between eleven cysteine residues in each hinge region of its two heavy chains.

The term “CH2 domain” refers to the portion of a heavy chain immunoglobulin molecule that extends, e.g., from about positions 244-360 in the Kabat numbering system (EU positions 231-340). The CH2 domain is unique in that it is not closely paired with another domain. Rather, two N-linked branched carbohydrate chains are interposed between the two CH2 domains of an intact native IgG molecule.

The term “CH3 domain” refers to the portion of a heavy chain immunoglobulin molecule that extends approximately 110 residues from N-terminus of the CH2 domain, e.g., from about positions 361-476 of the Kabat numbering system (EU positions 341-445). The CH3 domain typically forms the C-terminal portion of the antibody. In some immunoglobulins, however, additional domains may extend from CH3 domain to form the C-terminal portion of the molecule (e.g. the CH4 domain in the μ chain of IgM and the ε chain of IgE).

The term “CL domain” refers to the constant region domain of an immunoglobulin light chain that extends, e.g. from about Kabat position 107A-216. The CL domain is adjacent to the VL domain.

The term “Fc region” refers to the portion of a heavy chain constant region beginning in the hinge region just upstream of the papain cleavage site (i.e. residue 216 in IgG, taking the first residue of heavy chain constant region to be 114) and ending at the C-terminus of the antibody. Accordingly, a complete Fc region comprises at least a hinge domain, a CH2 domain, and a CH3 domain. The term Fc region encompasses a dimer of Fc regions as found in a native antibody molecule.

The term “linker” or “linking moiety” refers to moieties which are capable of linking one or more hinge regions of hinge-containing molecules to each other. Linkers may also link a hinge to one or more additional hinges. The linking moiety may be selected such that it is cleavable (e.g., enzymatically cleavable or pH-sensitive) or non-cleavable. The linker may include a spacer portion formed from a polymer, such as a hydrocarbon polymer, a peptidic polymer, a glycan polymer, polyethylene glycol, or other linkage and/or polymer spacers. The spacer may have functional groups on one or both of its ends, such as a bioorthogonal click chemistry group (e.g. DBCO or an azide) or a moiety that reacts with sulfhydryl groups (e.g. dibromomaleimide).

Examples of linkers include:

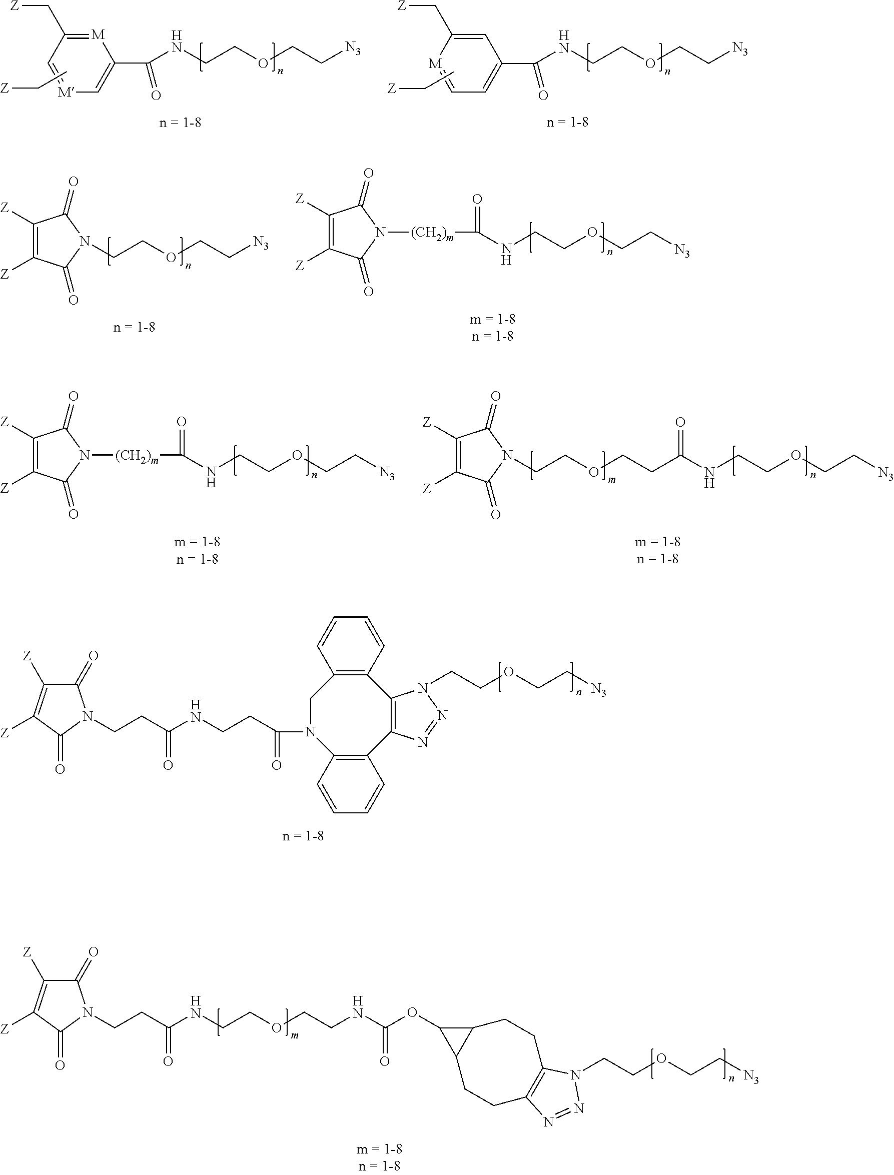

wherein N₃ is —N═N═N;

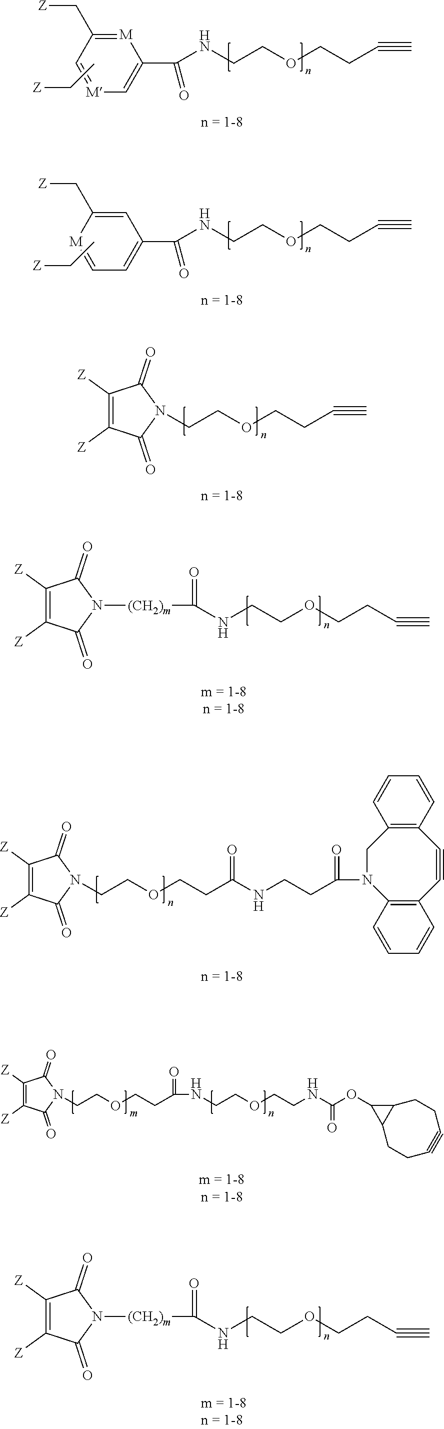

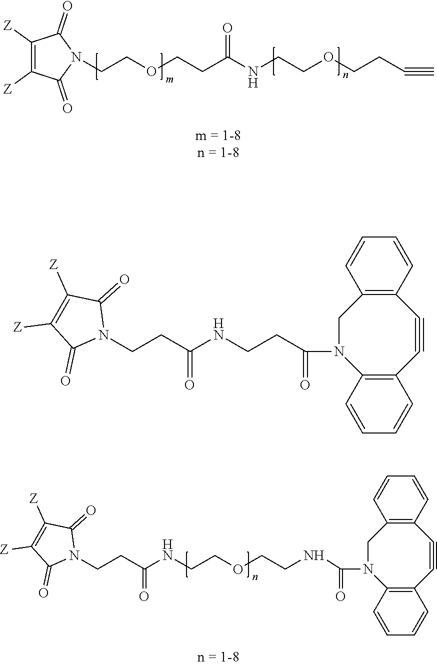

The term “bioorthogonal” or “orthogonal” with respect to a moiety or functional group on a linker, refers to the moiety not being naturally occurring, but that is able to react with a complementary moiety or functional group without interfering with native biochemical processes of an organism. This can include small organic molecules as well as enzymes and enzyme substrates. For example, copper-free click chemistry may be used to join two moieties together without using a copper catalyst that is toxic to cells and organisms. Orthogonal moieties include, but are not limited to: aldehydes, ketones, aminooxy groups, hydrazine, selenocysteine, dibenzocyclooctyl groups, trans-cyclooctene, alkynes, azides, tetrazine, and olefins. Orthogonal moiety reactions may include, but are not limited to: ketone/hydroxylamine condensation, Diels-Alder cycloaddition, Staudinger ligation, cross-metathesis, Pd-catalyzed cross coupling, strain-promoted alkyne-azide cycloadditions, strain-promoted alkyne-nitrone cycloaddition, copper-catalyzed alkyne-azide cycloaddition, photo-click cycloaddition, and 1,2-aminothiol-CBT condensations.

The term “sulfhydryl-reactive moiety” refers to a chemical moiety that forms a covalent bond with a sulfhydryl group on a protein residue (e.g. a reduced cysteine residue of a disulfide bond-forming cysteine pair in a hinge). Such sulfhydryl-reactive moieties may include, but are not limited to: maleimide, halogen-acetyl, halogen-substituted arene, pyridyldithiol, dibromomaleimide, dibromopyridazinediones, dithiophenolmaleimide, vinyl sulfone, methylsulfone-containing heteroaromatic, bis-sulfone, arsenic-thiol, dibromo-xylene, dihalogen-aromatic, dichloroacetone, 3-aryl-propiolonitriles, and derivatives or analogs thereof. In certain embodiments, the sulfhydryl-reactive moiety is bifunctional and forms covalent bonds with both reduced cysteine residues of a disulfide bond-forming cysteine pair in a hinge.

The term “hinge-joining moiety” refers to the adduct of a sulfhydryl-reactive moiety and a sulfhydryl group of a disulfide bond-forming cysteine pair in a hinge. Or the hinge-joining moiety is bifunctional and is covalently bonded to both cysteine residues of a disulfide bond-forming cysteine pair in a hinge.

The term “reducing agent” refers to any chemical agent that can reduce a disulfide bond between two hinge regions of a protein to sulfhydryls, without reducing other disulfide bonds in another portion of the protein (or an associated protein molecule) that are protected or partially protected by other amino acid residues of a polypeptide. Exemplary reducing agent include, but are not limited to: tris(2-carboxyethyl)phosphine (TCEP); dithiothreitol (DTT); mercaptoethanol; mercaptoethylamine; cysteine; glutathione; thiophenol; and benzeneselenol.

The term “click chemistry” refers to chemistry tailored to generate covalent bonds quickly and reliably by joining small units comprising reactive groups together (see H. C. Kolb, M. G. Finn and K. B. Sharpless (2001). Click Chemistry: Diverse Chemical Function from a Few Good Reactions. Angewandte Chemie International Edition 40 (11): 2004-2021. Click chemistry does not refer to a specific reaction, but to a concept including, but not limited to, reactions that mimic reactions found in nature. Click chemistry reactions can be modular, wide in scope, give high chemical yields, generate inoffensive byproducts, are stereospecific, exhibit a large thermodynamic driving force to favor a reaction with a single reaction product, and/or can be carried out under physiological conditions. In certain embodiments, a click chemistry reaction exhibits high atom economy, can be carried out under simple reaction conditions, use readily available starting materials and reagents, uses no toxic solvents or uses a solvent that is benign or easily removed (preferably water), and/or provides simple product isolation by non-chromatographic methods (crystallization or distillation). In certain embodiments, the click chemistry reaction is a [2+3] dipolar cycloaddition. In certain embodiments, the click chemistry reaction is a Diels-Alder cycloaddition.

The term “click chemistry handle” refers to a reactant, or a reactive group, that can partake in a click chemistry reaction. Exemplary click chemistry handles are demonstrated in U.S. Patent Publication 2013/0266512, which is incorporated by reference herein. For example, a strained alkyne, e.g., a cyclooctyne, is a click chemistry handle, since it can partake in a strain-promoted cycloaddition (see, e.g., Table 1 below). In general, click chemistry reactions require at least two molecules comprising click chemistry handles that can react with each other. Such click chemistry handle pairs that are reactive with each other are sometimes referred to herein as partner click chemistry handles. For example, an azide is a partner click chemistry handle to a cyclooctyne or any other alkyne. Exemplary click chemistry handles suitable for use according to some embodiments are described herein, for example, in Tables 1 and 2. In certain embodiments, the click chemistry partners are a conjugated diene and an optionally substituted alkene. In other embodiments, the click chemistry partners are optionally substituted tetrazine (Tz) and optionally substituted trans-cyclooctene (TCO). Tz and TCO react with each other in a reverse-electron demand Diels-Alder cycloaddition reaction (see e.g., Blackman et al., “The Tetrazine Ligation: Fast Bioconjugation based on Inverse-electron-demand Diels-Alder Reactivity.” J. Am. Chem. Soc. 2008; 130, 13518-13519). In other embodiments, the click chemistry partners are an optionally substituted alkyne and an optionally substituted azide. For example, a difluorinated cyclooctyne, a dibenzocyclooctyne, a biarylazacyclooctynone, or a cyclopropyl-fused bicyclononyne can be paired with an azide as a click chemistry pair. In other embodiments, the click chemistry partners are reactive dienes and suitable tetrazine dienophiles. For example, TCO, norbornene, or biscyclononene can be paired with a suitable tetrazine dienophile as a click chemistry pair. In yet other embodiments, tetrazoles can act as latent sources of nitrile imines, which can pair with unactivated alkenes in the presence of ultraviolet light to create a click chemistry pair, termed a “photo-click” chemistry pair. The click chemistry pair may also be a cysteine and a maleimide. For example, the cysteine from a peptide (e.g., GGGC) may be reacted with a maleimide that is associated with a chelating agent (e.g., NOTA). Other suitable click chemistry handles are known to those of skill in the art (Table 1; Spicer et al., “Selective chemical protein modification.” Nature Communications. 2014; 5:4740). For two molecules to be conjugated via click chemistry, the click chemistry handles of the molecules have to be reactive with each other, for example, in that the reactive moiety of one of the click chemistry handles can react with the reactive moiety of the second click chemistry handle to form a covalent bond. Such reactive pairs of click chemistry handles are well known to those of skill in the art and include, but are not limited to, those described in Table 1 below.

TABLE 1
Exemplary click chemistry handles and reactions.

| | | Exemplary rate |
|---|---|---|
| | | constant (M⁻¹s⁻¹) |
| 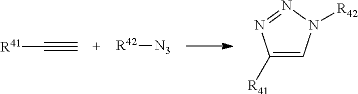 | 1,3-dipolar cycloaddition | 1 × 10^{-3 a} |
| 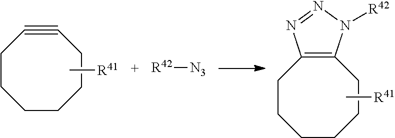 | strain-promoted cycloaddition | |
| 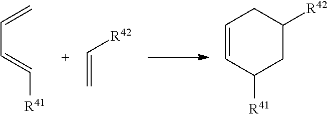 | Diels-Alder reaction | |
| 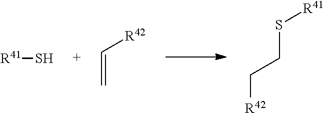 | Thiol-ene reaction | |
| 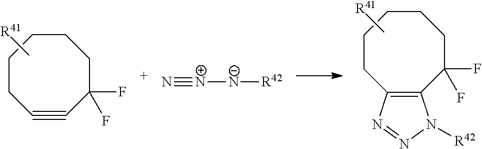 | Strain-promoted cycloaddition | 8 × 10^{-2 a} |
| 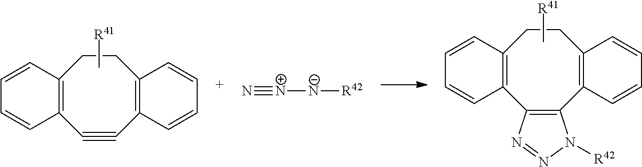 | Strain-promoted cycloaddition | 2.3^{a} |
| 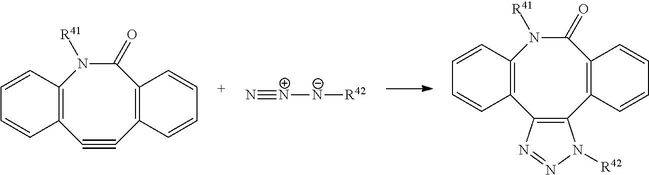 | Strain-promoted cycloaddition | 1^{a} |
| 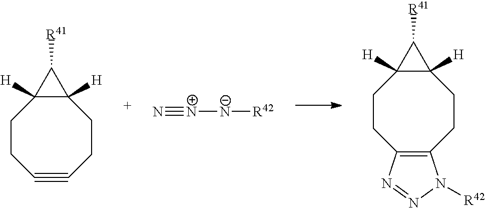 | Strain-promoted cycloaddition | 0.1^{a} |
| 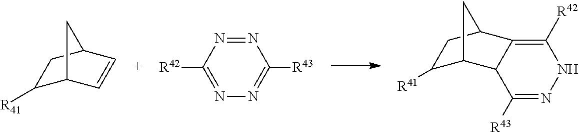 | Inverse-electron demand Diels- Alder (IEDDA) | 9^{a} |
| 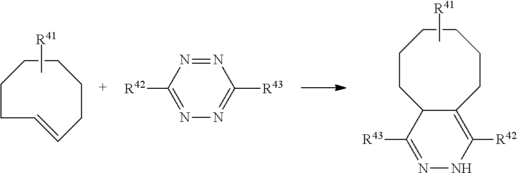 | Inverse-electron demand Diels- Alder (IEDDA) | 17,500^{a} 35,000^{b} |
| 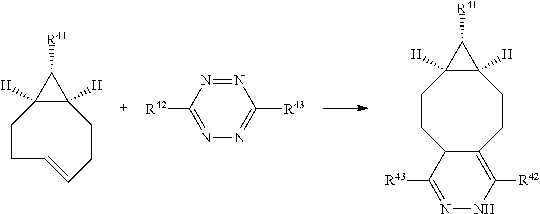 | Inverse-electron demand Diels- Alder (IEDDA) | >50,000^{a} 880^{b} |
| 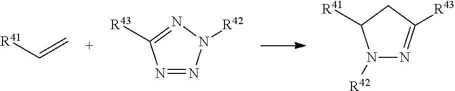 | 1,3-dipolar cycloaddition (“photo-click”) | 0.9^{a} |
| 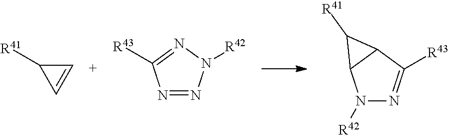 | 1,3-dipolar cycloaddition (“photo-click”) | 58^{a} |

Each of R⁴¹, R⁴², and R⁴³ is indpendently hydrogen, optionally suLDtituted aliphatic, optionally suLDtituted heteroaliphatic, optionally suLDtituted aryl, optionally suLDtituted heteroaryl, or optionally suLDtituted heterocyclyl.
^{a}Exemplary rate constant for small-molecule models.
^{b}Exemplary on-protein rate constant.

In certain embodiments, click chemistry handles used can react to form covalent bonds in the absence of a metal catalyst, making them compatible for use in biological systems. Such click chemistry handles are well known to those of skill in the art and include the click chemistry handles described in Becer, Hoogenboom, and Schubert (see Table 2 below), “Click Chemistry beyond Metal-Catalyzed Cycloaddition,” Angewandte Chemie International Edition (2009) 48: 4900-4908:

TABLE 2
Exemplary click chemistry reactions.

| | Reagent A | Reagent B | Mechanism | Notes on reaction^{[a]} |
|---|---|---|---|---|
| 0 | azide | alkyne | Cu-catalyzed [3 + 2] | 2 h at 60° C. in H₂O |
| | | | azide-alkyne cycloaddition | |
| | | | (CuAAC) | |
| 1 | azide | cyclooctyne | strain-promoted [3 + 2] azide-alkyne cycloaddition | 1 h at RT |
| | | | (SPAAC) | |
| 2 | azide | activated | [3 + 2] Huisgen cycloaddition | 4 h at 50° C. |
| | | alkyne | | |
| 3 | azide | electron-deficient alkyne | [3 + 2] cycloaddittion | 12 h at RT in H₂O |
| 4 | azide | aryne | [3 + 2] cycloaddition | 4 h at RT in THF with crown ether or |
| | | | | 24 h at RT in CH₃CN |
| 5 | tetrazine | alkene | Diels-Alder retro-[4 + 2] cycloaddition | 40 min at 25° C. (100% yield) |
| 6 | tetrazole | alkene | 1,3-dipolar cycloaddition | N₂ is the only by-product |
| | | | (photoclick) | few min UV irradiation and then overnight |
| | | | | at 4° C. |
| 7 | dithioester | diene | hetero-Diels-Alder cycloaddition | 10 min at RT |
| 8 | anthracene | maleimide | [4 + 2] Diels-Alder reaction | 2 days at reflux in toluene |
| 9 | thiol | alkene | radical addition | 30 min UV (quantitative conv.) or |
| | | | (thio click) | 24 h UV irradiation (>96%) |
| 10 | thiol | enone | Michael addition | 24 h at RT in CH₃CN |
| 11 | thiol | maleimide | Michael addition | 1 h at 40° C. in THF or |
| | | | | 16 h at RT in dioxane |
| 12 | thiol | para-fluoro | nucleophilic substitution | overnight at RT in DMF or |
| | | | | 60 min at 40° C. in DMF |
| 13 | amine | para-fluoro | nucleophilic substitution | 20 min MW at 95° C. in NMP as soivent |

^{[a]}RT = room temperature, DMF = N,N-dimethylformamide, NMP = N-methylpyrolidone, THF = tetrahydrofuran, CH₃CN = acetonitrile.

The term “click chemistry reaction product” refers to a chemical structure that results from reacting two click chemistry handles. For example, the click chemistry reaction product of an azide and DBCO is a triazole moiety.

The term “binding moiety” refers to a molecule (e.g., a protein, an antibody, antibody fragment, or receptor fragment) that contains at least one binding site which is responsible for selectively binding to a target antigen of interest (e.g. a human antigen). Exemplary binding sites include an antibody variable domain, a ligand binding site of a receptor, a receptor binding site of a ligand, or a cell-adhesion molecule. In certain aspects, the binding moieties of the disclosure comprise multiple (e.g., two, three, four, or more) binding sites. Exemplary binding moieties may include, but are not limited to: antibodies; Fab fragments; diabodies; immunoadhesins; bi-specific T cell engagers (BiTEs; Amgen Inc., Thousand Oaks, Calif., USA); dual-affinity re-targeting proteins (DARTs; Macrogenics Inc., Rockville, Md., USA); TandAb molecules (Affimed NV, Heidelberg, DE); and dock-and-lock molecules (Immunomedics Inc., Morris Plains, N.J.). BiTEs are fusion proteins that include two single-chain variable fragments (scFv) on a single polypeptide chain. One of the scFv fragments binds to a CD3 receptor, and the other scFv fragment recognizes another antigen (such as a tumor cell molecule). DARTs are fusion proteins having two Fv fragments, with each Fv fragment formed by the association of a VL partner on one chain with a VH partner on the second chain in a VLA-VHB VLB-VHA configuration. TandAb molecules are fusion proteins formed by linking four variable domains of heavy and light chains (VH and VL) from two different Fv in a single polypeptide. This creates two tandem diabodies. Dock-and-lock molecules are fusion proteins that use the interaction of cyclic AMP-dependent protein kinase (PKA) and A-kinase anchoring protein (AKAP). One fusion protein having a PKA domain (e.g. an antibody with a certain specificity) and another fusion protein having the AKAP domain (e.g. an antibody with a different specificity) can be associated by binding of the PKA and AKAP domains.

The term “cetuximab IgG2” refers to a derivative of the anti-EGFR antibody cetuximab (CAS number: 205923-56-4) in which the heavy chain constant region is replaced with a human IgG2 constant region.

The term “trastuzamab IgG2” refers to a derivative of the anti-HER2 antibody trastuzamab (CAS number: 180288-69-1) in which the heavy chain constant region is replaced with a human IgG2 constant region.

The term “conjugation partner” refers to a molecule that can be conjugated to hinge-containing molecules (such as antibodies) that are functionalized with a linker molecule. Such conjugation partners may include another hinge-containing molecule (e.g. an antibody or antibody fragment), a peptide, a receptor, a protein ligand, or an enzyme (e.g. β-glucosidase, β-glucouronidase), or a small molecule (e.g. a fluorescent label or a chemotherapeutic agent). Conjugation partners may also include molecules used to treat and reduce disease, such as enzymes that catalyze reaction with a prodrug. Antibody-directed enzyme prodrug therapy (ADEPT) utilizes an antibody paired with an enzyme that recognizes a particular antigen (such as a tumor antigen). The associated enzyme (e.g. β-glucosidase, β-glucouronidase) then reacts with a prodrug administered to a subject and converts the prodrug (e.g. Amygdalin or doxorubicin) into an active drug at the disease site (such as a solid tumor or tumor cell). This allows lower amounts of drug to be administered and achieving a higher specificity of drug action.

### II. Hinge-Containing Molecules and Methods of Derivatizing and Conjugating the Same

This disclosure provides methods for functionalizing hinge-containing molecules. The hinge-containing molecules include a hinge having cysteine residues that form multiple disulfide bonds. The disclosed method includes reducing an interchain disulfide bond in a hinge of the hinge-containing molecule to produce two sulfhydryls, the hinge having at least two interchain disulfide bonds. The hinge-containing molecule is then contacted by a linker, in order to join the linker to the reduced disulfide bond. The linker comprises a sulfhydryl-reactive moiety and a bioorthogonal moiety, such that each of the sulfhydryl groups of the reduced interchain disulfide bond are conjugated to the sulfhydryl-reactive moiety to form a covalently-linked adduct with the hinge. After contacting the hinge-containing molecule with the linker, the hinge has at least one interchain disulfide bond. The bioorthogonal moiety is then available for reaction with another bioorthogonal moiety attached to a conjugation partner, such as an antibody, an enzyme, or a small molecule.

The present disclosure also provides methods for conjugating two or more hinge-containing molecules. The methods include reducing an interchain disulfide bond in a first hinge of a first hinge-containing molecule to produce two sulfhydryls. The first hinge may be contacted with a first linker that includes a first sulfhydryl-reactive moiety and a first bioorthogonal moiety. Each of the sulfhydryl groups of the reduced first hinge is conjugated to the first sulfhydryl-reactive moiety to form a covalently-linked adduct with the first hinge-containing molecule. The methods also include reducing an interchain disulfide bond in a second hinge of a second hinge-containing molecule to produce two sulfhydryls. The second hinge is contacted with a second linker including a second sulfhydryl-reactive moiety and a second bioorthogonal moiety. Each of the sulfhydryl groups of the reduced second hinge are then conjugated to the second sulfhydryl-reactive moiety to form a covalently-linked adduct with the second hinge-containing molecule. The methods also include contacting the first hinge-containing molecule with the second hinge-containing molecule, such that the first and second bioorthogonal moieties form a covalently-linked adduct between the first hinge-containing molecule and the second hinge-containing molecule. After contacting the first and second hinge-containing molecule with the first and second linker, the first and second hinge each has at least one interchain disulfide bond. In certain embodiments, the first and second hinge-containing molecules are identical. In certain embodiments, the first and second hinge-containing molecules are different. In certain embodiments, the first and second hinge-containing molecules are trastuzumab IgG2 and cetuximab IgG2, respectively.

The present disclosure further provides hinge-containing molecules. The hinge-containing molecules may include a hinge that includes an interchain disulfide bond, as well as a linker molecule. The linker molecule may include a hinge-joining moiety that covalently links both cysteine residues of a disulfide bond-forming cysteine pair of the hinge, and a bioorthogonal moiety. The hinge of the hinge-containing molecule includes at least one interchain disulfide bond, in addition to the linker molecule.

The present disclosure further provides a composition that includes a first hinge-containing molecule and a second hinge-containing molecule. The first hinge-containing molecule includes a first hinge and an interchain disulfide bond, while the second hinge-containing molecule includes a second hinge and an interchain disulfide bond. The disclosed composition may also include a linker molecule that links the first and second hinge-containing molecules. The linker may include a first hinge-joining moiety that covalently links both cysteine residues of a disulfide bond-forming cysteine pair of the first hinge. The linker may also include a second hinge-joining moiety that covalently links both cysteine residues of a disulfide bond-forming cysteine pair of the second hinge. In certain embodiments, the first and second hinge-joining moieties may be the same type of moiety, or they may be different. The linker may also include a bioorthogonal moiety that covalently links the first and second hinge-containing molecules.

Any hinge-containing molecule may be derivatized or conjugated using the method and compositions disclosed herein. In certain embodiments, a hinge-containing molecule may be an antibody or an immunoadhesin, or combinations thereof. In certain embodiments, the hinge-containing molecule may be a full-length antibody, such as a monoclonal antibody against an antigen expressed in a cancer or other disease. Suitable pre-existing monoclonal antibodies include, without limitation, 3F8, 8H9, abagovomab, abciximab, abituzumab, abrilumab, actoxumab, adalimumab, adecatumumab, aducanumab, afasevikumab, afelimomab, afutuzumab, alacizumab pegol, ALD518, alemtuzumab, alirocumab, altumomab pentetate, amatuximab, anatumomab mafenatox, anetumab ravtansine, anifrolumab, anrukinzumab (IMA-638), apolizumab, arcitumomab. ascrinvacumab, aselizumab, atezolizumab, atinumab, atlizumab (tocilizumab), atorolimumab, avelumab, bapineuzumab, basiliximab, bavituximab, bectumomab, begelomab, belimumab, benralizumab, bertilimumab, besilesomab, bevacizumab, bezlotoxumab, biciromab, bimagrumab, bimekizumab, bivatuzumab mertansine, bleselumab, blinatumomab. blontuvetmab, blosozumab, bococizumab, brazikumab, brentuximab vedotin, briakinumab, brodalumab, brolucizumab, brontictuzumab, burosumab, cabiralizumab, canakinumab, cantuzumab mertansine, cantuzumab ravtansine, caplacizumab, capromab pendetide, carlumab, carotuximab, catumaxomab, cBR96-doxorubicin immunoconjugate, cedelizumab, cergutuzumab amunaleukin, certolizumab pegol, cetuximab, ch.14.18, citatuzumab bogatox, cixutumumab, clazakizumab, clenoliximab, clivatuzumab tetraxetan, codrituzumab, coltuximab ravtansine, conatumumab, concizumab, crenezumab, crotedumab, CR6261, dacetuzumab, daclizumab, dalotuzumab, dapirolizumab pegol, daratumumab, dectrekumab, demcizumab, denintuzumab mafodotin, denosumab, depatuxizumab mafodotin, derlotuximab biotin, detumomab, dinutuximab, diridavumab, domagrozumab, dorlimomab aritox, drozitumab, duligotumab, dupilumab, durvalumab, dusigitumab, ecromeximab, eculizumab, edobacomab, edrecolomab, efalizumab, efungumab, eldelumab, elgemtumab, elotuzumab, elsilimomab, emactuzumab, emibetuzumab, emicizumab, enavatuzumab, enfortumab vedotin, enlimomab pegol, enoblituzumab, enokizumab, enoticumab, ensituximab, epitumomab cituxetan, epratuzumab, erenumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, fibatuzumab, ficlatuzumab, figitumumab, firivumab, flanvotumab, fletikumab, fontolizumab, foralumab, foravirumab, fresolimumab, fulranumab, futuximab, galcanezumab, galiximab, ganitumab, gantenerumab, gavilimomab, gemtuzumab ozogamicin, gevokizumab, girentuximab, glembatumumab vedotin, golimumab, gomiliximab, guselkumab, ibalizumab, ibritumomab tiuxetan, icrucumab, idarucizumab, igovomab, IMAB362, imalumab, imciromab, imgatuzumab, inclacumab, indatuximab ravtansine, indusatumab vedotin, inebilizumab. infliximab, intetumumab, inolimomab, inotuzumab ozogamicin, ipilimumab, iratumumab, isatuximab, itolizumab, ixekizumab, keliximab, labetuzumab, lambrolizumab, lampalizumab, lanadelumab, landogrozumab, laprituximab emtansine, lebrikizumab, lemalesomab, lendalizumab, lenzilumab, lerdelimumab, lexatumumab, libivirumab, lifastuzumab vedotin, ligelizumab, lilotomab satetraxetan, lintuzumab, lirilumab, lodelcizumab, lokivetmab, lorvotuzumab mertansine, lucatumumab, lulizumab pegol, lumiliximab, lumretuzumab, mapatumumab, margetuximab, maslimomab, mavrilimumab, matuzumab, mepolizumab, metelimumab, milatuzumab, minretumomab, mirvetuximab soravtansine, mitumomab, mogamulizumab, monalizumab, morolimumab, motavizumab, moxetumomab pasudotox, muromonab-CD3, nacolomab tafenatox, namilumab, naptumomab estafenatox, naratuximab emtansine, narnatumab, natalizumab, navicixizumab, navivumab, nebacumab, necitumumab, nemolizumab, nerelimomab, nesvacumab, nimotuzumab, nivolumab, nofetumomab merpentan, obiltoxaximab, obinutuzumab, ocaratuzumab, ocrelizumab, odulimomab, ofatumumab, olaratumab, olokizumab, omalizumab, onartuzumab, ontuxizumab, opicinumab, oportuzumab monatox, oregovomab, orticumab, otelixizumab, otlertuzumab, oxelumab, ozanezumab, ozoralizumab. pagibaximab, palivizumab, pamrevlumab, panitumumab, pankomab, panobacumab, parsatuzumab, pascolizumab, pasotuxizumab, pateclizumab, patritumab, pembrolizumab, pemtumomab, perakizumab, pertuzumab, pexelizumab, pidilizumab, pinatuzumab vedotin, pintumomab, placulumab, plozalizumab, pogalizumab, polatuzumab vedotin, ponezumab, prezalizumab, priliximab, pritoxaximab, pritumumab, PRO 140, quilizumab, racotumomab, radretumab, rafivirumab, ralpancizumab, ramucirumab, ranibizumab, raxibacumab, refanezumab, regavirumab, reslizumab, rilotumumab, rinucumab, risankizumab, rituximab, rivabazumab pegol, robatumumab, roledumab, romosozumab, rontalizumab, rovalpituzumab tesirine, rovelizumab, ruplizumab, sacituzumab govitecan, samalizumab, sapelizumab, sarilumab, satumomab pendetide, secukinumab, seribantumab, setoxaximab, sevirumab, sibrotuzumab, SGN-CD19A, SGN-CD33A, sifalimumab, siltuximab, simtuzumab, siplizumab, sirukumab, sofituzumab vedotin, solanezumab, solitomab, sonepcizumab, sontuzumab, stamulumab, sulesomab, suvizumab, tabalumab, tacatuzumab tetraxetan, tadocizumab, talizumab, tamtuvetmab, tanezumab, taplitumomab paptox, tarextumab, tefibazumab, telimomab aritox, tenatumomab, teneliximab, teplizumab, teprotumumab, tesidolumab, tetulomab, tezepelumab, TGN1412, ticilimumab (tremelimumab), tildrakizumab, tigatuzumab, timolumab, tisotumab vedotin, TNX-650, tocilizumab (atlizumab), toralizumab, tosatoxumab, tositumomab, tovetumab, tralokinumab, trastuzumab, trastuzumab emtansine, TRLD07, tregalizumab, tremelimumab, trevogrumab, tucotuzumab celmoleukin, tuvirumab, ublituximab, ulocuplumab, urelumab, urtoxazumab, ustekinumab, utomilumab, vadastuximab talirine, vandortuzumab vedotin, vantictumab, vanucizumab, vapaliximab, varlilumab, vatelizumab, vedolizumab, veltuzumab, vepalimomab, vesencumab, visilizumab, vobarilizumab, volociximab, vorsetuzumab mafodotin, votumumab, xentuzumab, zalutumumab, zanolimumab, zatuximab, ziralimumab, and zolimomab aritox.

In certain embodiments, the hinge-containing molecule comprises a chimeric heavy chain constant region comprising an altered hinge and/or a hinge from a different isotype from one or more other regions of the heavy chain constant region. In certain embodiments, the hinge-containing molecule may be a derivative of a pre-existing therapeutic antibody or immunoadhesin that has an altered hinge relative to the pre-existing therapeutic antibody or immunoadhesin. In certain embodiments, the derivative comprises a hinge that has one or more amino acid substitutions relative to the pre-existing therapeutic antibody or immunoadhesin. In certain embodiments, the derivative comprises a hinge from a different Ig isotype relative to the pre-existing therapeutic antibody or immunoadhesin. In certain embodiments, the derivative comprises a hinge and a CH1 domain from a different Ig isotype relative to the pre-existing therapeutic antibody or immunoadhesin (e.g., where the hinge and a CH1 domain are from the same isotype; or where the hinge and a CH1 domain are each from different isotypes and each of the hinge and CH1 domain isotypes differ from the isotype of the therapeutic antibody or immunoadhesin). In certain embodiments, the derivative comprises a hinge and a CH2 domain from a different Ig isotype relative to the pre-existing therapeutic antibody or immunoadhesin (e.g., where the hinge and a CH2 domain are from the same isotype; or where the hinge and a CH2 domain are each from different isotypes and each of the hinge and CH2 domain isotypes differ from the isotype of the therapeutic antibody or immunoadhesin). In certain embodiments, the derivative comprises an Fc region from a different Ig isotype relative to the pre-existing therapeutic antibody or immunoadhesin. In certain embodiments, the derivative comprises an entire heavy chain constant region from a different Ig isotype relative to the pre-existing therapeutic antibody or immunoadhesin. In certain embodiments, the antibody is trastuzumab IgG2 or cetuximab IgG2, or a combination, thereof as described herein.

The hinge-containing molecule includes a binding moiety proximate to the hinge. For example, the binding moiety may be a Fab fragment, a diabody, an immunoadhesin, an Fcab molecule, a bi-specific T cell engager molecule (BiTE), a DART molecule, a TandAb molecule, or a dock-and-lock molecule. In certain embodiments, the binding moiety may be an antibody-like molecule (e.g., fibronectin domain), a T-cell receptor, an extracellular domain of a cell-surface receptor, a cell-adhesion molecule, or a ligand.

Reducing the interchain disulfide bond in the hinge is accomplished by using a reducing agent to preferentially reduce the interchain disulfide bond under conditions such that non-hinge disulfide bonds are not reduced. The hinge-containing molecule may include one or more non-hinge disulfide bonds, for example in the binding moiety, that are not reduced by the reducing agent. Any reducing agent that can preferentially reduce an interchain disulfide bond in the hinge without reducing non-hinge disulfide bonds can be employed in the methods disclosed herein. Suitable reducing agents include, without limitation, tris(2-carboxyethyl)phosphine (TCEP), dithiothreitol (DTT), mercaptoethanol, mercaptoethylamine, cysteine, glutathione, thiophenol, or benzeneselenol. In certain embodiments, the reducing agent is dithiothreitol (DTT) in certain embodiments.

Any sulfhydryl-reactive moiety that can react with an interchain disulfide bond in the hinge can be employed in the methods and molecules disclosed herein. In certain embodiments, the linkers disclosed herein comprise a bifunctional sulfhydryl-reactive moiety that reacts with both reduced cysteine residues of a disulfide bond-forming cysteine pair in a hinge. In certain embodiments of the methods and molecules disclosed herein, the sulfhydryl-reactive moiety of the linker may include a maleimide moiety, such as a dibromomaleimide (DBM) moiety that react with the reduced sulfhydryls of the hinge. In certain embodiments of hinge-containing molecules and compositions of this disclosure, a hinge-joining moiety of a hinge-containing molecule may include a product of a reaction between a sulfhydryl-reactive moiety and a sulfhydryl as a dithiolmaleimide (DTM) (the product of a reaction between dibromomaleimide and two sulfhydryls of a hinge).

Any bioorthogonal moiety can be employed in the methods and molecules disclosed herein. In certain embodiments, the bioorthogonal moiety of a linker may include a click chemistry handle. Exemplary click chemistry handles are set forth in Table 1 and 2 herein. Exemplary click chemistry handles include, but are not limited to an azide, a nitrone, a cyclooctyne, an aldehyde, a ketone, a tetrazine, a cyclooctene, a cyclooctyne, an isonitrile, a quadracyclane, or a nickel bis(dithiolene). In certain embodiments, the click chemistry handle may be an azide or a cyclooctyne, such as or dibenzylcyclooctyne (DBCO). The click chemistry handle may be chosen in order to react with a partner click chemistry handle on a conjugation partner (e.g. an antibody, a binding moiety, an enzyme, or a small molecule), so that the hinge-containing molecule is conjugated with the conjugation partner. For example, a full length antibody may be conjugated to the hinge-containing molecule to create a bispecific antibody.

The bioorthogonal moiety of a linker may include a click chemistry reaction product resulting from the reaction of two click chemistry handles. For example, in certain embodiments the click chemistry reaction product may be the reaction product of an azide and a cyclooctyne; the reaction product of an azide and an activated alkyne; the reaction product of an azide and an electron-deficient alkyne; the reaction product of an azide and an aryne; the reaction product of a tetrazine and an alkene; the reaction product of a tetrazole and an alkene; or the reaction product of a tetrazine and a cyclooctene. In certain embodiments, the click chemistry reaction product comprises the reaction product of an azide and a cyclooctyne.

In certain embodiments, the biorthogonal moiety of a linker may include a click chemistry reaction product resulting from a type of click chemistry reaction, such a strain-promoted [3+2] azide-alkyne cycloaddition, a [3+2] Huisgen cycloaddition, ad [3+2] cycloaddition, a Diels-Alder retro-[4+2] cycloaddition, or a photoclick addition.

### EXAMPLES

The following examples are meant to be illustrative and should not be construed as further limiting. The contents of the figures and all references, patents, and published patent applications cited throughout this application are expressly incorporated herein by reference.

### Example 1. LDAb Formation

It was theorized that LDAb formation could be enabled through reduction of the interchain disulfide bonds of two different native antibodies (such as in an IgG2 antibody), followed by conjugation with a dibromomaleimide-based (DBM) linker (e.g. containing either azide or dibenzylcyclooctyne (DBCO) groups), followed by attachment of the linker-associated antibodies using copper-free click chemistry (e.g. between the azide and DBCO groups). trastuzumab IgG2 and cetuximab IgG2 were used. Without being bound by theory, using IgG2 antibodies may result in more homogeneous conjugates, as the hinge contains four disulfide bonds. The additional disulfide bonds of IgG2 (in contrast with other immunoglobulins such as IgG1) may facilitate more selective reduction of the hinge-proximate disulfide bonds over the heavy-light chain disulfide pair.

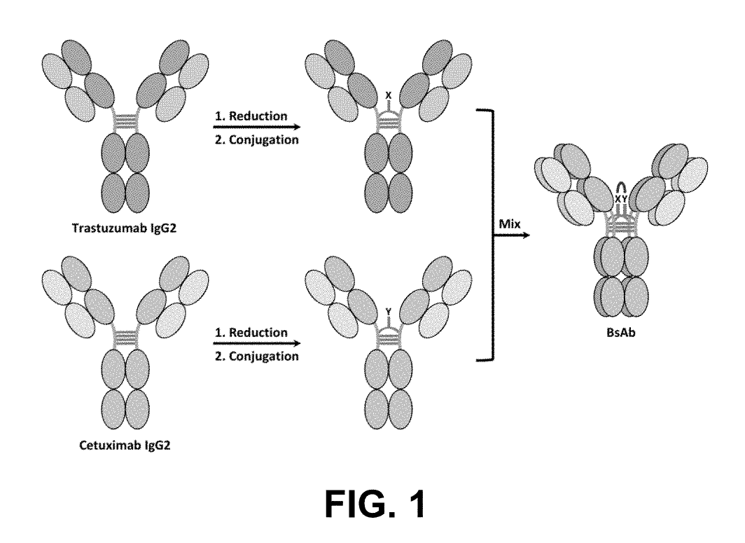

FIG. 1 shows a schematic diagram of the LDAb formation strategy. Trastuzumab IgG2 (αHER2) and cetuximab IgG2 (αEGFR), both having four disulfide bonds in the hinge, are subjected to reducing conditions to create free sulfhydryls from the disulfide bonds. Linkers are then added to react with the free sulfhydryls on the antibodies (“X” on the trastuzumab; “Y” on the cetuximab), replacing one of the disulfide bonds. The antibodies are then mixed together, allowing a click chemistry reaction between the “X” and “Y” groups of the trastuzumab and cetuximab antibodies, covalently attaching the native antibodies together.

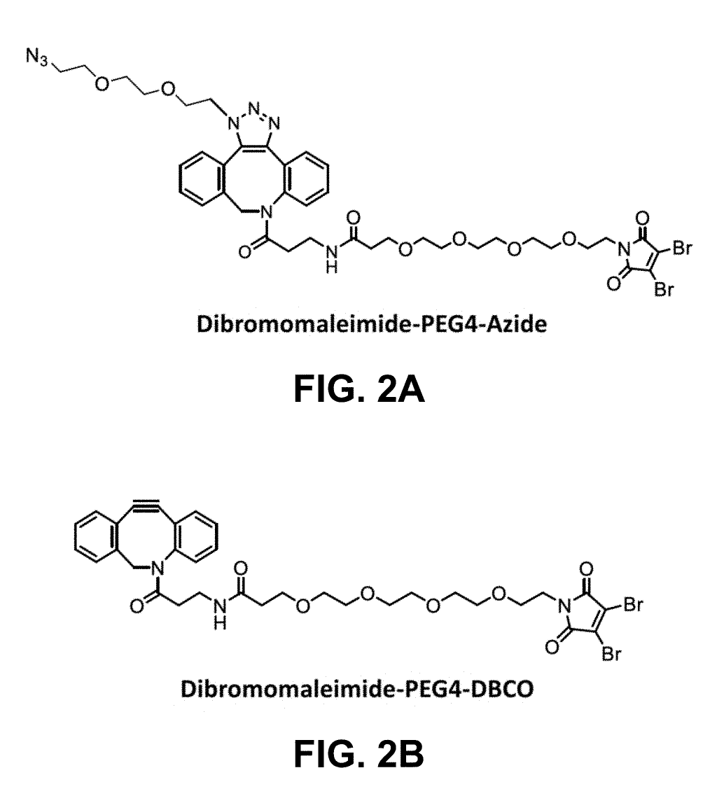

FIGS. 2A and 2B depict the linkers utilized. FIG. 2A is the chemical structure of dibromomaleimide-PEG4-azide (DBM-PEG4-azide). FIG. 2B shows the chemical structure of dibromomaleimide-PEG4-dibenzocyclooctyl (DBM-PEG4-DBCO). Each of the bromines on the DBM group on the linkers in FIGS. 2A and 2B participates in a substitution reaction with the thiols of a reduced disulfide bond in the hinge of an antibody. The DBCO and azide groups of the linkers react via a standard 1,3-dipolar cycloaddition to form a single linked unit. The azide linker was prepared in situ by reacting a DBCO-PEG4-DBM linker with 10 equivalents azido-PEG2-azide for 1 h at room temperature (RT).

### Materials and Methods

In these examples of LDAb formation, trastuzumab IgG2 and cetuximab IgG2 were conjugated with DBM-PEG4-azide- and DBM-PEG4-DBCO-bearing linkers, respectively, for the click reaction. Conditions were compared using 2, 5, and 10 equivalents of either tris(2-carboxyethyl)phosphine (TCEP) or dithiothreitol (DTT) to 5 mg/mL antibody for 1 h reduction at RT followed by conjugation with 10-15 equivalents dibromomaleimide linker overnight at RT. Excess linker was removed by centrifugal filtration. LDAb synthesis was initiated by mixing trastuzumab IgG2 azide and cetuximab IgG2 DBCO at 5 mg/mL for 24 h at 37° C.

Analysis of the reactions for synthesizing LDAb was performed by size-exclusion chromatography (SEC) HPLC. SEC was performed using an Agilent 1260 Infinity HPLC system (Agilent Technologies, Inc., Santa Clara, Calif., USA) with a TOSOH TSKgel SuperSW3000 (4.6 mm ID×30 cm, 4 μm) column (Tosoh Bioscience, Inc., King of Prussia, Pa., USA). The column was maintained at 25° C. utilizing Dulbecco's phosphate-buffered saline (DPLD) as mobile phase and isocratic elution over 20 min at 0.6 mL/min. Analytical analysis was executed using 100 μg sample. All data was analyzed using OpenLAB software.

Hydrophobic interaction chromatography (HIC) HPLC was employed to assess LDAb formation and purity. Analysis by HIC HPLC used a TOSOH TSKgel Butyl-NPR (4.6 mm ID×10 cm, 2.5 μm) column (Tosoh Bioscience, Inc., King of Prussia, Pa., USA) at 40° C. on an Agilent 1260 Infinity system (Agilent Technologies, Inc., Santa Clara, Calif., USA). Analytical runs were performed using 50 μg sample using a linear gradient of 0-50% B over 25 min at 0.6 mL/min: A=50 mM sodium phosphate+1 M ammonium sulfate (pH 7), B=50 mM sodium phosphate+25% isopropanol (pH 7). All data was analyzed using OpenLAB Software.

Characterization of conjugates and clicked products was also performed by sodium dodecyl sulfate polyacrylamide gel electrophoresis (SDS-PAGE). SDS-PAGE utilized NuPAGE Novex 3-8% Tris-Acetate Protein Gels with NuPAGE Tris-Acetate SDS Running Buffer (Thermo Fisher Scientific, Inc., Waltham, Mass., USA) in a XCell SureLock Mini electrophoresis system (125 V for 1 h) (Thermo Fisher Scientific, Inc., Waltham, Mass., USA). All samples (3.5 μg) included NuPAGE LDS Sample Buffer, and reduced gels included NuPAGE Sample Reducing Agent. Samples were heating to 95° C. for 5 min prior to loading. HiMark Unstained Protein Standard (10 μL) (Thermo Fisher Scientific, Inc., Waltham, Mass., USA) was used for analysis of high molecular weight proteins. Gels were fixed for 5 min and stained with SYPRO Ruby Protein Gel Stain (Thermo Fisher Scientific, Inc., Waltham, Mass., USA) following the recommended procedures. Imaging was performed with a Bio-Rad ChemiDoc MP System (Bio-Rad, Inc., Hercules, Calif., USA) using the SYPRO Ruby filter and analyzed by Image Lab Software.

A Biacore T200 system (GE Lifesciences, Inc., Pittsburgh, Pa., USA) was used to measure the affinity of LDAb. Anti-human Fc antibody was immobilized on a CMS sensor chip to approximately 8,000 RU using standard NHS/EDC coupling methodology. Antibodies (˜2 μg/mL) were captured for 60 s at a flow rate 10 μL/min. Recombinant HER2/His or EGFR/His was serially diluted in running buffer (HLD-EP+). All measurements were conducted with a flow rate of 30 μL/min. Surfaces were regenerated with 3M MgCl₂ (from human antibody kit) for 60 s. A 1:1 (Langmuir) binding model was used to fit the data.

The ability for LDAb to simultaneously bind HER2 and EGFR antigens was analyzed using an Octet RED system (ForteBio, Menlo Park, Calif., USA). All analysis was performed at 25° C. Biosensors were first activated by standard NHS/EDC coupling methodology, and HER2 antigen was immobilized at 10 μg/mL in 200 μL of 10 mM sodium acetate buffer (pH 5) with 1 M ethanolamine (pH 8.5) quenching. After baseline acquisition, LDAb (20 μg/mL) association and dissociation was measured in Dulbecco's phosphate buffered saline (DPLD) buffer. Association and dissociation with EGFR (20 μg/mL) was then measured in DPLD buffer to determine if LDAb could engage both HER2 and EGFR antigens.

Specific binding of IgG2 antibodies to human cancer cells was assayed by flow cytometry. HCC1954, MDA-MB-468, MCF7, and MDA-MB-435 cells in exponential growth were harvested with enzyme-free Cell Dissociation Buffer (Gibco/Thermo Fisher Scientific Inc., Waltham, Mass., USA), resuspended in fluorescence-activated cell sorting (FACS) buffer (PLD+2% FLD+0.01% NaN3) and transferred to V-Bottom 96 well-plates (50,000 cells/well). Cells were incubated on ice for 30 min with serial dilutions of IgG2 antibodies in FACS buffer. After 2 washes in FACS buffer, a 1:1,000 dilution of phycoerythrin (PE)-conjugated anti-human IgG (SouthernBiotech, Inc., Birmingham, Ala., USA) was added and incubated for 20 min. Following a final wash, fluorescence intensity was measured on an Intellicyt High Throughput Flow Cytometer (HTFC) (Intellicyt, Inc., Albuquerque, N. Mex., USA). Data were analyzed using Graphpad Prism software and non-linear regression fit.

### Results

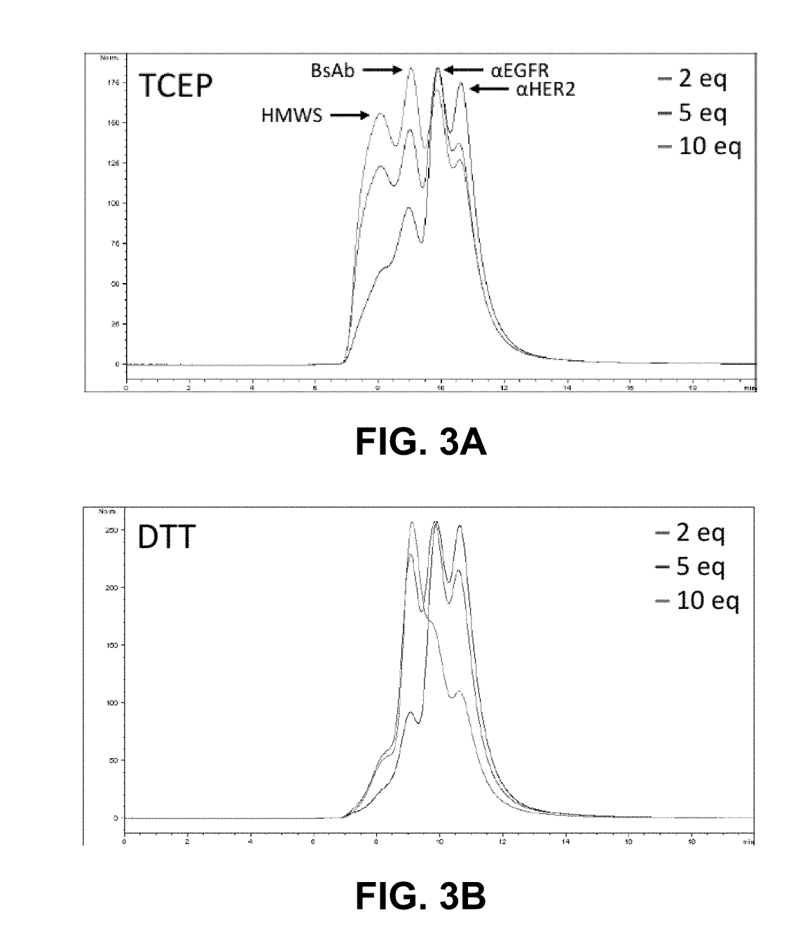

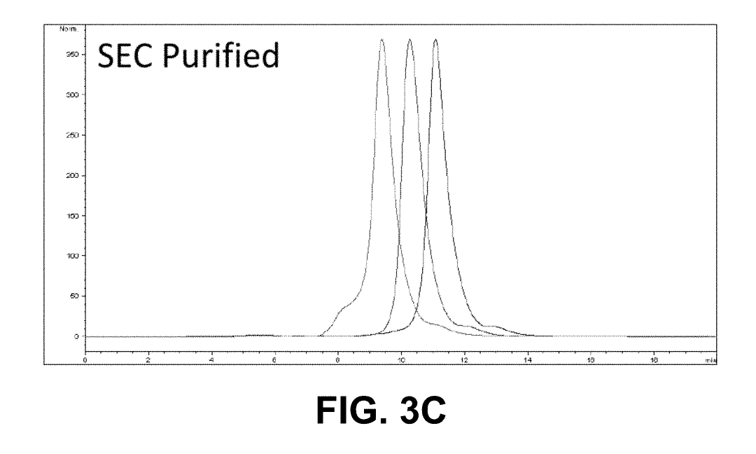

The analytical SEC column was capable of resolving the parent antibodies from the clicked products, as demonstrated in FIGS. 3A-3C. FIG. 3A shows a chromatogram for SEC HPLC of LDAb created using TCEP as a reducing agent. Peaks for the LDAb, anti-EGFR antibody, and anti-HER2 antibody are indicated by the arrows. FIG. 3B is a chromatogram for SEC HPLC of LDAb created using DTT as a reducing agent, and FIG. 3C is a chromatograph of purified LDAb, using SEC HPLC. A high molecular weight species (HMWS) peak (see arrow in FIG. 3A) was observed for both sets of TCEP and DTT reduction conditions; however, this peak was significantly decreased using the mild DTT reducing agent. LDAb peak was also observed with more product formed using increasing equivalents of reducing agent. Quantification of the LDAb and HMWS peaks revealed substantially improved LDAb formation for the DTT conditions. LDAb yield was at least 2-fold higher for DTT than TCEP when comparing the 10 equivalents condition, while HMWS was ˜3-fold lower.

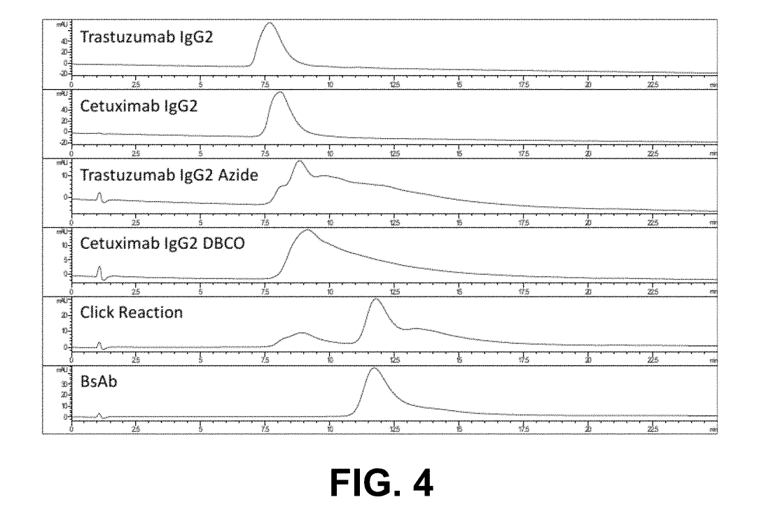

Formation of the LDAb antibody was also confirmed by HIC HPLC. FIG. 4 is a series of HIC HPLC chromatograms of untreated trastuzumab IgG2, untreated cetuximab IgG2, trastuzumab IgG2 azide, cetuximab IgG2-DBCO, the click reaction, and the purified LDAb. Both the trastuzumab and cetuximab IgG2 proteins showed a uniform peak. Conjugation using the methods described above yielded a shift in retention time that is consistent with increased hydrophobicity due to linker attachment (FIG. 4, “click reaction”). The azide and DBCO samples had a broadened rear peak likely caused by a distribution of conjugated products. SEC purification of the LDAb after click reaction effectively removed non-clicked and over-conjugated species.

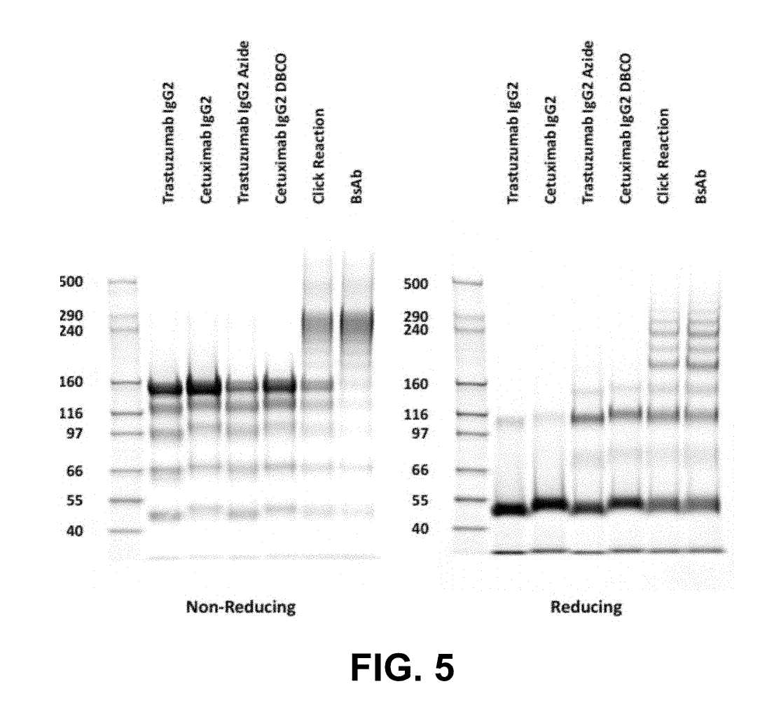

The major product was confirmed by SDS-PAGE to have the expected ˜300 kDa molecular weight for the αHER2×αEGFR LDAb (FIG. 5). FIG. 5 shows pictures of SDS-PAGE non-reducing and reducing gels, with lanes labeled for untreated trastuzumab IgG2, untreated cetuximab IgG2, trastuzumab IgG2 azide, cetuximab IgG2-DBCO, the click reaction, and the purified LDAb. Protein separation by non-reducing SDS-PAGE revealed a shift in molecular weight for the clicked product that was consistent with the expected ˜300 kDa LDAb. A small amount of non-clicked species remained in the click reaction sample as may be expected for incomplete disulfide crosslinking and/or copper-free click chemistry. The non-clicked species was efficiently removed from the LDAb sample by SEC purification. Similar results have been obtained using HIC purification. The reduced gel shows a predominant band in the trastuzumab IgG2 azide and cetuximab IgG2 DBCO lanes of ˜100 kDa, which would be expected for the desired inter-heavy chain-bridged conjugate. However, intra-heavy chain modification is also possible and would not alter the separation of these species on the gel as compared to parental IgG2 proteins. Minor bands are also apparent at ˜75 and 125 kDa that likely correspond to heavy-light chain (HL) and heavy-heavy-light chain (HHL) crosslinked species, respectively. Hence, the distribution of products in clicked samples. Nonetheless, selectivity for hinge reduction is apparent and results in a homogeneous molecular weight LDAb.

### Example 2. Antigen Recognition Analysis

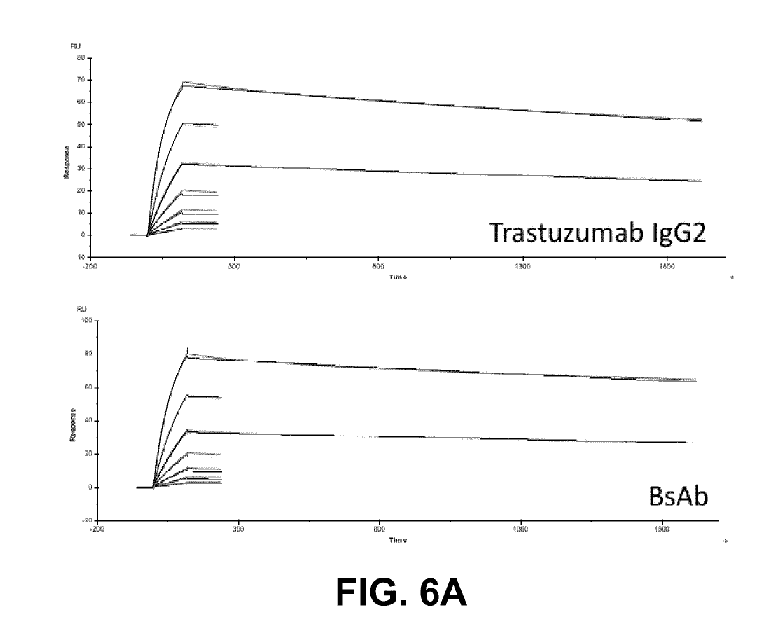

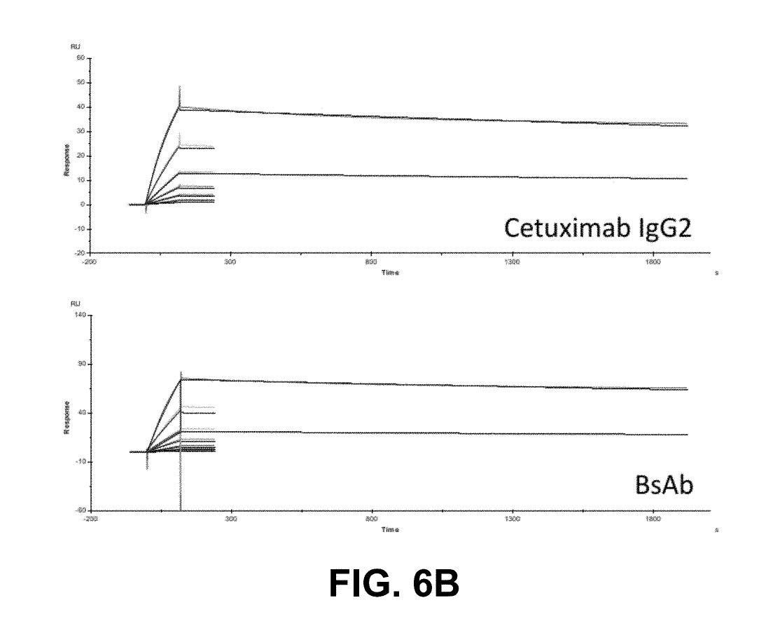

The ability of the αHER2×αEGFR LDAb to maintain antigen recognition by surface plasmon resonance (SPR) analysis was analyzed. FIG. 6A shows a surface plasmon resonance (SPR) graph for binding of the HER2 antigen by trastuzumab IgG2 (upper graph) and the LDAb (lower graph). FIG. 6B shows an SPR graph for binding of the EGFR antigen to cetuximab IgG2 (upper graph) and the LDAb (lower graph). K_{D} values for HER2 binding were determined to be 0.81 and 0.52 nM for trastuzumab IgG2 and LDAb, respectively. Similarly, K_{D} values for EFGR binding were determined to be 2.1 and 2.8 nM for cetuximab IgG2 and LDAb, respectively. Thus, antigen binding is not impaired by LDAb formation.

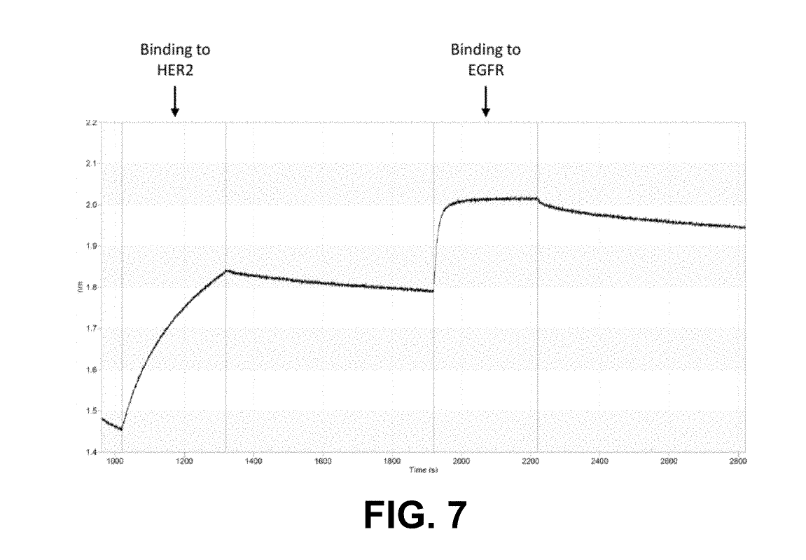

To assess if the LDAb could simultaneously engage both antigens, HER2 was first immobilized on the sensor followed by addition of LDAb then soluble EGFR. FIG. 7 shows a biolayer interferometry graph over time. The binding data shows that LDAb can indeed bind both antigens simultaneously; an arrow in FIG. 7 indicates binding of HER2 by the LDAb, and a second arrow indicates binding of EGFR by the LDAb.

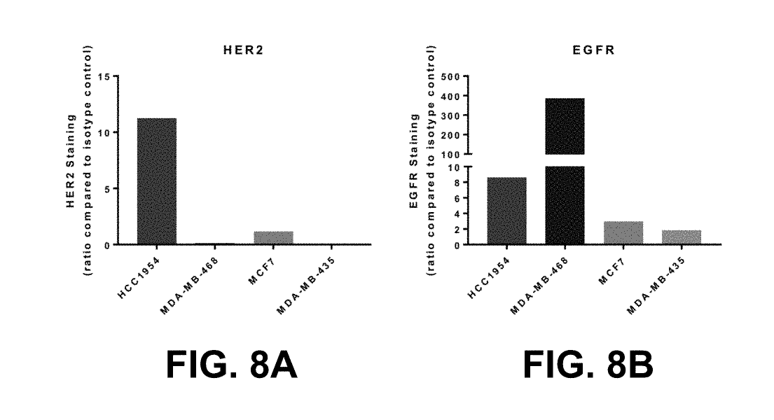

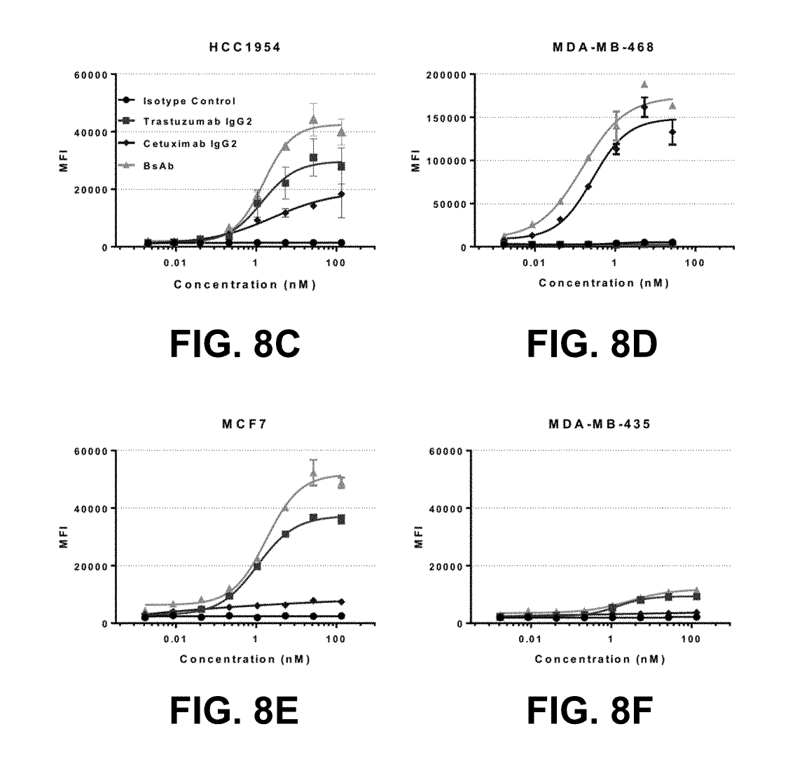

Binding of LDAb was further evaluated on cells using immunofluorescent staining and flow cytometry. FIGS. 8A-8B show the levels of expression of HER2 and EGFR on each cell line, as determined by immunofluorescent staining. Briefly, cells in V-bottom plates were incubated on ice for 20 min with 5 μL of ALEXA FLUOR 488-anti-human HER2 antibody (BioLegend, Inc., San Diego, Calif., USA) and/or with 5 μL of PE-anti-human EGFR antibody (BioLegend, Inc., San Diego, Calif., USA). Cells were then washed in FACS buffer, and staining was analyzed by high throughput flow cytometry (HTFC). FIG. 8A is a bar graph of the ratio of fluorescence relative to isotype control for four cell lines, indicating the level of HER2 staining for those cell lines. HER2 staining levels were highest in HCC1954 (showing a ratio-to-control level of about 10), and much lower in MCF7, with little to no HER2 staining detected in MDA-MB-468 cells and MDA-MB-435. FIG. 8B is a bar graph of the ratio of fluorescence for the same cell lines, indicating the level of EGFR staining for those cell lines. MDA-MB-468 cells showed the highest levels of EGFR staining (a ratio-to-control level of about 400), with HCC1954 cells showing more moderate EGFR staining levels (ratio-to-control level of about 8). MCF7 cells and MDA-MB-435 cells showed lower levels of EGFR staining (ratio-to-control level of about 2).

The MCF7 cells did show more balanced expression between the two antigens. Lastly, no significant binding was observed with MDA-MB-435 control cells. HER2 expression was highest for HCC1954 cells, which also showed some EGFR expression. However, MDA-MB-468 cells expressed only high levels of EGFR. MCF7 cells had a lower expression of both receptors, whereas MDA-MB-231 had very little expression of either receptor.

Results for the individual cells lines stained with trastuzumab IgG2, cetuximab IgG2, and the LDAb are shown in FIGS. 8C-8F. FIG. 8C is a line graph for cell line HCC1954, FIG. 8D is a line graph for cell line MDA-MB-468, FIG. 8E is a line graph for cell line MCF7, and FIG. 8F is a line graph for cell line MDA-MB-435. Data points for FIGS. 8C-8F are shown as the median fluorescence intensity (MFI) of positively labeled cells +/−standard error. Binding by trastuzumab IgG2 and cetuximab IgG2 to their respective antigens was generally consistent with the expression profiles determined in FIG. 8A. However, similar MFI values were obtained with trastuzumab IgG2 and LDAb for HCC1954 and MCF7 cells despite higher HER2 expression on the HCC1954 cell line. This demonstrates that maximal HER2 binding is sufficient with MCF7 HER2 levels.

EGFR binding on MDA-MB-468 cells was only observed for cetuximab IgG2 and LDAb, with no trastuzumab IgG2 binding detected. Cetuximab IgG2 did not bind as strongly on the lower EGFR expressing HCC1954 and MCF7 cells. Both antibodies had no significant binding to MDA-MB-435 cells with only basal HER2 binding detected. Trastuzumab IgG2 and LDAb bound to HER2-dominant HCC-1954 with an ˜3 nM affinity. Similarly, LDAb showed comparable binding to cetuximab-IgG2 on EGFR-dominant MDA-MB-468 cells with an affinity of ˜0.2 nM. A slight increase in maximal binding was noted with MCF7 cells; this is despite the low expression of both receptors. Collectively, flow cytometry analysis has demonstrated that LDAb can recognize both HER2 and EGFR expressing tumor cells with similar binding potency as the parental IgG2 antibodies, and binding to cells expressing both antigens may potentially improve binding.

The examples demonstrate robust bioorthogonal strategy for synthesizing IgG2 LDALD. Disulfide bridging with DBM linkers allows installation of azide and DBCO functionalities for click chemistry. This work emphasizes the importance of optimizing the reduction conditions to maximize LDAb yield and homogeneity. An αHER2×αEGFR bispecific antibody was generated using this method which demonstrated excellent antigen binding by both SPR and cell-based flow cytometry measurements. This platform offers the advantage of preserving the Fc region as compared to other scFv, Fab, and F(ab′)₂ formats. Preserved valency of the chemically-produced LDAb will also confer tremendous advantage for targeting antigens in a variety of diseases.

We claim:

1. A method for functionalizing a hinge-containing antibody molecule, comprising:
(a) reducing an antibody interchain disulfide bond in a hinge region of an antibody molecule having at least two interchain disulfide bonds, to produce two sulfhydryl groups in reduced interchain disulfide bonds; and
(b) contacting the antibody molecule with two sulhydryls with a linker, wherein the linker comprises a sulfhydryl-reactive moiety and a bioorthogonal moiety, whereby each of the sulfhydryl groups of the reduced interchain disulfide bond are conjugated to the sulfhydryl-reactive moiety to form a covalently-linked adduct with the hinge; wherein after contacting the hinge-containing molecule with the linker, the hinge has at least one interchain disulfide bond.

2. The method for functionalizing a hinge-containing antibody molecule of claim 1, wherein the antibody molecule is selected from the group consisting of IgG2 and a F(ab′)₂.

3. The method for functionalizing a hinge-containing antibody molecule of claim 1, wherein reducing is accomplished by using a reducing agent selected from the group consisting of: tris(2-carboxyethyl)phosphine (TCEP), dithiothreitol (DTT), mercaptoethanol, mercaptoethylamine, cysteine, glutathione, thiophenol, and benzeneselenol.

4. The method for functionalizing a hinge-containing antibody molecule of claim 3, wherein the reducing agent is dithiothreitol (DTT).

5. The method for functionalizing a hinge-containing antibody molecule of claim 1, wherein the sulfhydryl-reactive moiety comprises a dibromomaleimide (DBM) moiety.

6. The method for functionalizing a hinge-containing antibody molecule of claim 1, wherein the bioorthogonal moiety comprises a click chemistry handle, comprising one or more moieties selected from the group consisting of: an azide; a nitrone; a cyclooctyne; an aldehyde; a ketone; a tetrazine; a cyclooctene; an isonitrile; a quadracyclane; a nickel bis(dithiolene), and a dibenzylcyclooctyne (DBCO).

7. A method for conjugating two hinge-containing antibody molecules, comprising:
(a) reducing an interchain disulfide bond in a first hinge of a first hinge-containing antibody molecule to produce two sulfhydryls;
(b) contacting the first hinge with a first linker comprising a first sulfhydryl-reactive moiety and a first bioorthogonal moiety, such that each of the sulfhydryl groups of the reduced first hinge is conjugated to the first sulfhydryl-reactive moiety to form a covalently-linked adduct with the first hinge-containing molecule;
(c) reducing an interchain disulfide bond in a second hinge of a second hinge-containing antibody molecule to produce two sulfhydryls; contacting the second hinge with a second linker comprising a second sulfhydryl-reactive moiety and a second bioorthogonal moiety, such that each of the sulfhydryl groups of the reduced second hinge is conjugated to the second sulfhydryl-reactive moiety to form a covalently-linked adduct with the second hinge-containing molecule; and
(d) contacting the first and second hinge-containing antibody molecules such that the first and second bioorthogonal moieties form a covalently-linked adduct between the first hinge-containing molecule and the second hinge-containing molecule, wherein after contacting the first and second hinge-containing molecule with the first and second linker, the first and second hinge each has at least one interchain disulfide bond.

8. The method for conjugating two hinge-containing antibody molecules of claim 7, wherein the first or second hinge-containing antibody molecule is selected from the group consisting of a full-length antibody, an IgG2, and a F(ab′)₂.

9. The method for conjugating two hinge-containing antibody molecules of claim 7, wherein the reducing agent is selected from the group consisting of: tris(2-carboxyethyl)phosphine (TCEP), dithiothreitol (DTT), mercaptoethanol, mercaptoethylamine, cysteine, glutathione, thiophenol, and benzeneselenol.

10. The method for conjugating two hinge-containing antibody molecules of claim 7, wherein the sulfhydryl-reducing moiety comprises a dibromomaleimide (DBM) moiety.

11. The method for conjugating two hinge-containing antibody molecules of claim 7, wherein the bioorthogonal moiety comprises a click chemistry handle, wherein the click chemistry handle comprises one or more moieties selected from the group consisting of: an azide; a nitrone; a cyclooctyne; an aldehyde; a ketone; a tetrazine; a cyclooctene; an isonitrile; a quadracyclane; a nickel bis(dithiolene), and a dibenzylcyclooctyne (DBCO).

12. An IgG2 class antibody or F(ab′)2 fragment thereof, having a hinge region hinge-containing molecule comprising: a hinge comprising an interchain disulfide bond; and a linker molecule comprising a hinge-joining moiety that covalently links both cysteine residues of a disulfide bond-forming cysteine pair of the hinge, and a bioorthogonal moiety, wherein, the hinge has at least one interchain disulfide bond.

13. A composition comprising: a first hinge-containing antibody molecule comprising a first hinge and an interchain disulfide bond; a second hinge-containing antibody molecule comprising a second hinge and an interchain disulfide bond; and a linker molecule comprising a first hinge-joining moiety that covalently links both cysteine residues of a disulfide bond-forming cysteine pair of the first hinge, a second hinge-joining moiety that covalently links both cysteine residues of a disulfide bond-forming cysteine pair of the second hinge, and a bioorthogonal moiety that covalently links the first and second hinge-containing molecules.
